(19) United States
(12) Patent Application Publication
(10) Pub. No.: US 2011/0104082 A1
(43) Pub. Date: May 5, 2011

(54) ENHANCED PHOTO PROTECTION

(75) Inventors: Jack Polonka, Peekskill, NY (US); Luis Roberto Misso, Stratford, CT (US)

(73) Assignee: CONOPCO, INC., D/B/A UNILEVER, Englewood Cliffs, NJ (US)

(21) Appl. No.: 12/611,942

(22) Filed: Nov. 4, 2009

(51) Int. Cl. A61K 8/30; A61Q 17/04

(52) U.S. Cl. 424/59

(57) ABSTRACT

A cosmetic composition is provided which includes from 1 to 5% of a water-insoluble UVA organic sunscreen agent having a λₘₐₓ between 330 and 380 nm and a photo protective enhancing agent including a monomeric organic amide, imide or nitrile of molecular weight no higher than 500 and having a dielectric constant ranging from 6 to 20, and a carrier which includes a crystalline gel structurant system.

### BACKGROUND OF THE INVENTION

1. Field of the Invention

The invention concerns cosmetic compositions with enhanced photoprotection properties.

2. The Related Art

Many people dislike northern climates. There is a longing to bask in the warmth of the sun. Days at the beach find us in swimsuit attire. Many seek to turn their pale winter skin into a bronzed appearance. Others of naturally darker skin simply enjoy the refreshment of the seashore. Without protection from harmful ultraviolet radiation damage, these pleasures can turn into premature aging. Skin can loose elasticity and wrinkles appear in the premature aging process. Radiation can promote erythemal damage, can cause photo allergic reactions, and is implicated in skin cancers.

Protective measures are necessary. Lotions and creams formulated with sunscreens can shield against ultraviolet damaging radiation. The extent of protection varies widely.

Numerous ultraviolet photo protective chemicals are known. Nonetheless, only a small number of sunscreen agents are both commercially available and approved by regulatory authorities. A need exists to operate with known approved commercial sunscreen agents yet formulating them to achieve more than their expected level of photo protection.

Representative of the art is U.S. Pat. No. 5,961,961 (Dobkowski et al.) which describes enhancing the photo protective effect by utilizing relatively large particle size titanium dioxide coupled with an organic sunscreen agent. Representative organic sunscreen agents include Benzophenone-3, octyl salicylate, octyl methoxycinnamate and 2-phenylbenzimidazole-5-sulphonic acid. Although this technology is useful, a need still exists to achieve higher efficacy for photo protective cosmetics employing standard commercial sunscreen agents.

### SUMMARY OF THE INVENTION

A cosmetic composition is provided which includes:
(i) from 1 to 5 weight % of a water-insoluble organic UV-A sunscreen agent having a λₘₐₓ between 330 and 380 nm;
(ii) a photo protective enhancing agent comprising a monomeric organic compound selected from the group consisting of an amide, an imide and a nitrile, the compound having a molecular weight less than 500, and a dielectric constant ranging from 6 to 20; and
(iii) a crystalline gel structurant system as a carrier comprising a surfactant and a co-surfactant, the surfactant comprising a C₁-C₂₀₀ polyethoxy or polypropoxy alcohol ester of a C₁₀-C₂₂ fatty acid and the co-surfactant comprising a mixture of a C₁₀-C₂₂ fatty alcohol, a glycerol ester of a C₁₀-C₂₂ fatty acid and a C₁₀-C₂₂ unesterified fatty acid.

### DETAILED DESCRIPTION OF THE INVENTION

Now it has been found that the photo protective properties of organic UV-A sunscreen agents in cosmetic compositions can be improved by incorporating a photo protective enhancing agent selected from monomeric organic amides, imides or nitriles. These compounds require a molecular weight of no higher than 500 and having a dielectric constant ranging from 6 to 20.

Cosmetic compositions of the present invention will include as a first element a water-insoluble organic UV-A sunscreen agent having a λₘₐₓ between 330 and 380 nm. Particularly the λₘₐₓ will range from 340 to 360 nm, and optimally be 360 nm. In this category of sunscreen agent, the preferred materials are Avobenzone (available as Parsol 1789®) and benzophenone-3 (also known as Oxybenzone) and combinations thereof.

Amounts of the water-insoluble UV-A sunscreen agent may range from 1 to 5%, optimally from 2 to 4% by weight of the composition.

Advantageously, the composition in one embodiment may include a cocktail of further photo protective agents. For instance, besides the water-insoluble organic UVA sunscreen agent, there may also be provided a water-insoluble UVB sunscreen agent having a λₘₐₓ at 280-320 nm.

A large variety of substances may be utilized as the UV-B sunscreen agent. Illustrative are 2-ethylhexyl p-methoxycinnamate, octyldimethyl p-aminobenzoic acid, digalloyltrioleate, ethyl 4-[bis(hydroxypropyl)]aminobenzoate, 2-ethylhexyl-2-cyano-3,3-diphenylacrylate, octylsalicylate, glyceryl p-aminobenzoate, 3,3,5-trimethylcyclohexylsalicylate, methylanthranilate, p-dimethylaminobenzoic acid or aminobenzoate, 2-ethylhexyl p-dimethylaminobenzoate, bis-ethylhexyloxyphenol methoxyphenol triazine, methylene bis-benzotriazolyl tetramethylbutylphenol, dimethicodiethylbenzal malonate, isoamyl methoxycinnamate, octyl triazone, and mixtures thereof.

Amounts of the water-insoluble UV-B sunscreen agent may range from 1 to 8%, preferably from 3 to 6%, and optimally about 5% by weight of the composition. Most preferred is octyl salicylate.

Advantageously but not necessarily the amount of water-insoluble UV-A to UV-B sunscreen agent may range from about 1:5 to 1:1, more preferably from 1:3 to 1:1, and optimally from 3:5 to 4:5 by weight of the composition.

A third type of photo protection that may be present is a water-soluble sunscreen agent having a λₘₐₓ between 280 and 400 nm. Especially useful for this purpose is 2-phenylbenzimidazole-5-sulfonic acid and salt forms. Amounts of the water-soluble sunscreen agent may range from 1 to 4%, preferably from 2 to 3%, and optimally about 3% by weight of the composition.

A key feature of the present invention is a photo protective enhancing agent. This agent may be a monomeric organic material selected from the group consisting of amides, imides and nitriles, all of which require a molecular weight no higher than 500, preferably no higher than 300, optimally no higher than 200.

Amounts of the photo protective enhancing agent may range from 0.001 to 10%, preferably from 0.01 to 5%, more preferably from 0.1 to 1%, and optimally from 0.3% to 0.5% by weight of the cosmetic composition.

Illustrative photo protective enhancing agents according to the present invention may have a amide structure of formula (I) as follows:

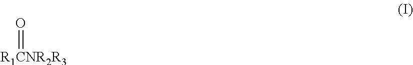

wherein R₂ and R₃ each independently are selected from the group consisting of hydrogen, alkyl, cycloalkyl, alkenyl phenyl, aryl, heteroaryl, hydroxyalkyl and mixtures thereof, and two of R₁, R₂ and R₃ may be linked to form a ring; or an imide structure of formula (II) as follows:

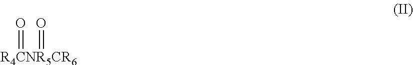

wherein R₄, R₅ and R₆ each independently are selected from the group consisting of hydrogen, alkyl, cycloalkyl, alkenyl, phenyl, aryl, heteroaryl, hydroalkyl and mixtures thereof, and two of R₄, R₅ and R₆ may be linked to form a ring; or a nitrile structure of formula (III) as follows:

R₇—CN (III)

wherein R₇ is selected from the group consisting of alkyl, cycloalkyl, alkenyl, phenyl, aryl, heteroalkyl, hydroxyalkyl and mixtures thereof.

Whenever any two radicals link to form a ring, the ring may, but need not necessarily, be selected from the group consisting of cycloalkyl, aryl and heteroalkyl.

Examples of amides of structure (I) include dimethyl decamide, ethylhexyl decamide, dimethyl lauramide, diethyl hexamide, diisopropyl lauramide, dimethyl benzamide, diisopropanolamide, dihexylacetamide, butylcyclohexamide, pyridinyl acetamide, dimethyl octylamide and combinations thereof. Illustrative materials according to the imide structural formula (II) include butyl isopropyl phthalimide, butyl phthalimide, dioctyl phthalimide, di-t-butyl phthalimide, bis(acetamide), N-methyl dibenzimide and combinations thereof. Illustrative substances according to the nitrile structural formula (III) include octyl nitrile, phenyl nitrile, dicyanobenzene, cyclohexyl nitrile, 2-hydroxy-8-cyanooctane, and mixtures thereof.

The photo protective enhancing agents according to this invention will have a dielectric constant ranging from 6 to 20, preferably from 6.5 to 18, more preferably from 7 to 15, and optimally from 7.5 to 12.

Compositions of this invention may include fatty acid alkanolamides such as stearamides but these are not for purposes of this invention considered to be the monomeric organic compounds identified as photo protective enhancing agents. Stearamides when present may be at levels from 0.001 to 10%, preferably from 0.05 to 2%, and more preferably from 0.1 to 0.5% by weight of the composition.

Cosmetic compositions of the present invention may be in cream or lotion form. These will feature a cosmetically acceptable carrier.

Carriers may be present in amounts ranging from about 5 to about 98%, preferably from about 20 to about 95%, optimally from about 40 to about 80% by weight of the cosmetic compositions. Water is the most common carrier for this invention. Oily carriers in the presence of water and an emulsifier will form emulsion systems as carriers. These systems may either be water-in-oil or oil-in-water emulsions. Besides water, suitable carrier classes include fatty acids, silicones, polyhydric alcohols, fatty alcohols, hydrocarbons, triglycerides and thickening powders.

Fatty acids may be selected from stearic acid, oleic acid, linoleic acid, linolinic acid, lauric acid, myristic acid, palmitic acid, behenic acid and mixtures thereof. Amounts may range from 1 to 50%, preferably from 8 to 30%, and optimally from 10 to 25% by weight of the composition.

Concentrations of the silicone may range from about 5% to about 60%, more preferably from about 5% to about 40%, by weight of the composition. These silicone fluids may be organic, silicone-containing or fluorine-containing, volatile or non-volatile, polar or non-polar.

Particularly preferred volatile silicone oils are cyclic volatile silicones wherein the repeating unit ranges from about 3 to about 5; and linear silicones wherein the repeating unit ranges from about 1 to about 7. Highly preferred examples of volatile silicone oils include cyclomethicones of varying viscosities, e.g., Dow Corning 200, Dow Corning 244, Dow Corning 245, Dow Corning 344, and Dow Corning 345, (commercially available from Dow Corning Corp.); SF-1204 and SF-1202 Silicone Fluids, GE 7207 and 7158 (commercially available from G.E. Silicones) and SWS-03314 (commercially available from SWS Silicones Corp.

Hydrocarbons may be useful as cosmetically acceptable carriers for compositions of this invention. They may include mineral oil, petrolatum and polyalpha-olefins. Examples of preferred volatile hydrocarbons include polydecanes such as isododecane and isodecane (e.g., Permethyl-99A which is available from Presperse Inc.) and the C7-C8 through C12-C15 isoparaffins (such as the Isopar Series available from Exxon Chemicals).

Polyhydric alcohols may serve as carriers. Illustrative of this group are propylyene glycol, dipropylene glycol, polypropylene glycol, polyethylene glycol, sorbitol, hydroxypropyl sorbitol, hexylene glycol, 1,3-butylene glycol, isoprene glycol, ethoxylated glycerol, propoxylated glycerol and mixtures thereof. Most preferred is glycerol known also as glycerin.

Fatty alcohols may also be useful carriers. The term “fatty” refers to carbon chain lengths ranging from 10 to 30 carbon atoms. Illustrative of this category are lauryl alcohol, cetyl alcohol, stearyl alcohol, isostearyl alcohol and combinations thereof.

Triglycerides are another group of materials useful as carriers. Illustrative but not limiting are sunflower seed oil, cotton oil, canola oil, soybean oil, castor oil, borage oil, olive oil, shea butter, jojoba oil and mixtures thereof. Mono- and di-glycerides may also be useful. Illustrative of these categories are glyceryl monostearate and glyceryl distearate.

The carriers can comprise one or more thickening agents, preferably from about 0.05% to about 10%, more preferably from about 0.1% to about 5%, and even more preferably from about 0.25% to about 4%, by weight for the composition.

In compositions of the present invention, the carrier is either exclusively or in part a crystalline gel structurant system. The crystalline gel structurant system includes a surfactant and a co-surfactant. The co-surfactant ordinarily will be essentially nonionic.

Nonionic type crystalline gel structurant systems will include a surfactant and a co-surfactant. Preferred nonionic structurant surfactants are C₁-C₂₀₀ esters of C₁₀-C₂₂ fatty acid. Esters of the fatty acid preferably are polyol esters such as C₂-C₃ alkoxylated alcohol esters. Among these are the polyethoxy, polypropoxy and block polyethyoxy/polypropoxy alcohol esters. Particularly preferred are such esters as PEG-100 stearate, PEG-20 stearate, PEG-80 laurate, PEG-20 laurate, PEG-100 palmitate, PEG-20 palmitate and combinations thereof.

The co-surfactant of a nonionic structurant typically may be a combination of a C₁₀-C₂₂ fatty alcohol, glyceryl esters of a C₁₀-C₂₂ fatty acid, and a C₁₀-C₂₂ unesterified fatty acid. Relative amounts of the ester to the alcohol may range from about 100:1 to about 1:100, preferably from about 50:1 to about 1:50, and optimally from about 3:1 to about 1:3 by weight. Relative amounts of the combination of glyceryl ester and fatty alcohol to unesterified fatty acid may range from about 100:1 to about 1:100, preferably from about 50:1 to about 1:50, and optimally from about 3:1 to about 1:3 by weight. Typical fatty alcohols include behenyl alcohol, stearyl alcohol, cetyl alcohol, myristyl alcohol, lauryl alcohol, oleyl alcohol and combinations thereof.

The relative amount of surfactant and co-surfactant in a nonionic structurant may range from about 50:1 to about 1:50, preferably from about 10:1 to about 1:10, and optimally from about 3:1 to about 1:3 by weight.

Cosmetic compositions of the present invention may contain a variety of optional components to enhance physical properties and performance.

The optional components, when incorporated into the cosmetic compositions, should be suitable for use in contact with human keratinous tissue without undue toxicity, incompatibility, instability, allergic response, and the like within the scope of sound judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these classes include: abrasives, absorbents, aesthetic components such as fragrances, pigments, colorings/colorants, essential oils, skin sensates, astringents, etc. (e.g. clove oil, menthol, camphor, eucalyptus oil, eugenol, menthyl lactate, witch hazel distillate), anti-acne agents, anti-caking agents, antifoaming agents, antimicrobial agents, antioxidants, biological additives, buffering agents, bulking agents, chelating agents, chemical additives, colorants, cosmetic astringents, cosmetic biocides, denaturants, drug astringents, external analgesics, film forming polymers, opacifying agents, pH adjusters, propellants, reducing agents, sequestrants, skin bleaching and lightening agents, skin conditioning agents, skin soothing and/or healing agents and derivatives, skin treating agents, thickeners, and vitamins and derivatives thereof.

A safe and effective amount of an anti-oxidant/radical scavenger may be added in amounts from about 0.01% to about 10%, more preferably from about 0.1% to about 5% by weight of the composition.

Anti-oxidants/radical scavengers may be employed such as ascorbic acid (vitamin C) and its salts, ascorbyl esters of fatty acids, ascorbic acid derivatives (e.g. magnesium ascorbyl phosphate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolor®), amines (e.g. N,N-diethylhydroxylamine, amino-guanidine), nordihydroguaiaretic acid, bioflavonoids, amino acids, cassia plant and extracts, silymarin, tea extracts, and grape skin/seed extracts. Preferred anti-oxidants/radical scavengers are selected from esters of tocopherol, more preferably tocopherol acetate.

The cosmetic compositions may optionally comprise a flavonoid compound. Flavonoids are disclosed in U.S. Pat. Nos. 5,686,082 and 5,686,367 herein incorporated by reference. Examples of flavonoids particularly suitable flavones, isoflavones, coumarins, chromones, discoumarols, chromanones, chromanols, isomers (e.g. cis/trans isomers) thereof, and mixtures thereof.

Preferred for use are flavones and isoflavones, in particular daidzein (7,4′-dihydroxy isoflavone), genistein (5,7,4′-trihydroxy isoflavone), equol (7,4′-dihydroxy isoflavan), 5,7-dihydroxy-4′-methoxy isoflavone, soy isoflavones (a mixture extracted from soy), and mixtures thereof. Flavonoid compounds useful herein are commercially available from a number of sources, e.g., Indofine Chemical Company, Inc., Stearloids, Inc., and Aldrich Chemical Company, Inc. The herein described flavonoid compounds are preferably present in from about 0.01% to about 20%, more preferably from about 0.1% to about 10%, and even more preferably from about 0.5% to about 5% by weight.

Anti-inflammatory agents useful herein include allantoin and compounds of the Licorice (the plant genus/species Glycyrrhiza glabra) family, including glycyrrhetic acid, glycyrrhizic acid, and derivatives thereof (e.g. salts and esters).

The compositions may comprise a tanning active. When present, it is preferable that the compositions comprise from about 0.1% to about 20%, more preferably from about 2% to about 7% by weight of the composition. A preferred tanning active is dihydroxyacetone.

The compositions may comprise a skin lightening agent. When used, the compositions preferably comprise from about 0.1% to about 10%, more preferably from about 0.2% to about 5%, also preferably from about 0.5% to about 2%, by weight of the composition, of a skin lightening agent. Suitable skin lightening agents include niacinamide, kojic acid, arbutin, tranexamic acid, placental extract, ascorbic acid and derivatives thereof (e.g. magnesium ascorbyl phosphate, sodium ascorbyl phosphate, ascorbyl glucoside, and ascorbyl tetraisopalmitates). Other skin lightening materials suitable for use herein include Actiwhite® (Cognis), Emblica® (Rona), Azeloglicina (Sinerga) and extracts (e.g. mulberry extract).

The compositions may comprise an antimicrobial or antifungal active. Such actives are capable of destroying microbes, preventing the development of microbes or preventing the pathogenic action of microbes. A safe and effective amount of an antimicrobial or antifungal active may be added to the present compositions, preferably, from about 0.001% to about 10%, more preferably from about 0.01% to about 5%, and even more preferably from about 0.05% to about 2% by weight of the composition.

Preferred examples of these actives include those selected from the group consisting of salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, phytic acid, N-acetyl-L-cystein, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, ciclopirox, lidocaine hydrochloride, clotrimazole, climbazole, miconazole, ketoconazole, neocycin sulfate, and mixtures thereof.

The compositions may comprise a conditioning agent selected from the group consisting of humectants, moisturizers, or skin conditioners. A variety of these materials can be employed and each can be present at a level of from about 0.01% to about 40%, more preferably from about 0.1% to about 30%, and even more preferably from about 0.5% to about 15% by weight of the composition. These materials include, but are not limited to, guanidine; urea; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); polyhydroxy compounds such as sorbitol, mannitol, glycerol, hexanetriol, butanetriol, propylene glycol, butylene glycol and hexylene glycol; polyethylene glycols; sugars and starch derivatives (e.g. alkoxylated glucose, fructose, sucrose, trehalose); hyaluronic acid; lactamide monoethanolamine; acetamide monoethanolamine; sucrose polyester; petrolatum; and mixtures thereof.

The cosmetic compositions include but are not limited to lotions, milks, mousses, serums, sprays, aerosols, foams, sticks, pencils, gels, creams and ointments. The compositions may also be applied via a woven or nonwoven synthetic and/or natural fibered textile (wipe or towelette).

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

All documents referred to herein, including all patents, patent applications, and printed publications, are hereby incorporated by reference in their entirety in this disclosure.

The following examples will more fully illustrate the embodiments of this invention. All parts, percentages and proportions referred to herein and in the appended claims are by weight unless otherwise illustrated.

### EXAMPLE

Photo protective effects were evaluated on a model system wherein the lamellar oil phase of an aqueous emulsion had the formula outlined in Table I. Weight percentages are on the basis of the total emulsion composition.

TABLE I

| Component | Weight % |
|---|---|
| Stearic Acid | 1.58 |
| Glycol Stearate/Stearamide AMP | 1.74 |
| Glyceryl Monostearate | 0.81 |
| Cetyl Alcohol | 0.47 |
| PEG-100 Stearate | 1.50 |
| Avobenzone | 3.00 |
| Octyl Salicylate | 5.00 |
| 2-Phenylbenzimidazole-5-sulfonic acid (in aqueous phase) | 1.00 |

### Procedure

### SPF Measurements

Sun protection factor (SPF) was measured in vitro using an Optometrics SPF 290 instrument. The test procedure required calibration of the monochrometer and sample stage of the Optometrics SPF 290 instrument. Thereafter the instrument was calibrated with a blank sample quartz plate (10 cm×10 cm and 3 mm thickness). Calibration zeros the UV detector. Formulas were applied and spread uniformly onto a plate to leave a film of 2 mg/cm². The film was left to dry for 30 minutes. Subsequently an SPF reading was taken on the dried film using three measurements on different parts of the coated quartz plate and recording an average value.

MPF is equivalent to the SPF value at a specific wavelength. For the present experiments the wavelength is the peak maximum at 305 to 360 nm.

### Results

The photo protective enhancing agents evaluated were butyl/isopropyl phthalimide and dimethyl decamide (DMDA). Dielectric constant values for the phthalimide and DMDA were respectively 7.5 and 12.

TABLE II

| | | UV Absorption Data | | |
|---|---|---|---|---|
| | | | Intensity | Intensity |
| | Enhancing Agent | In-vitro | λₘₐₓ = 305 nm | λₘₐₓ = 360 nm |
| Sample | (Weight %) | SPF | (MPF) | (MPF) |
| 1 | None | 25.4 | 53.3 | 18.2 |
| 2 | 0.1% Phthalimide | 25.9 | 48.7 | 25.2 |
| 3 | 0.5% Phthalimide | 24.3 | 50.3 | 27.5 |
| 4 | 0.1% DMDA | 23.8 | 49.1 | 25.7 |
| 5 | 0.5% DMDA | 28.3 | 52.3 | 33.2 |

The primary effect of the enhancing agents were upon increasing intensities of the λₘₐₓ 360 nm. For instance, Sample No. 1 is the base formula without any photo protective enhancing agent present. The intensity at λₘₐₓ 360 nm was 18.2. With only 0.1% of either phthalimide or DMDA, the intensity rose to over 25.

What is claimed is:

1. A cosmetic composition comprising:
(i) from 1 to 5 weight % of a water-insoluble organic UV-A sunscreen agent having a λₘₐₓ between 330 and 380 nm;
(ii) a photo protective enhancing agent comprising a monomeric organic compound selected from the group consisting of an amide, an imide and a nitrile, the compound having a molecular weight no higher than 500, and a dielectric constant ranging from 6 to 20; and
(iii) a crystalline gel structurant system as a carrier comprising a surfactant and a co-surfactant, the surfactant comprising a C₁-C₂₀₀ polyethoxy or polypropoxy alcohol ester of a C₁₀-C₂₂ fatty acid and the co-surfactant comprising a mixture of a C₁₀-C₂₂ fatty alcohol, a glycerol ester of a C₁₀-C₂₂ fatty acid and a C₁₀-C₂₂ unesterified fatty acid.

2. The composition according to claim 1 wherein the photo protective enhancing agent has a structure for the amide, imide and nitrile of respective structures (I), (II) and (III) as follows: 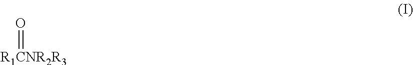
wherein R₁, R₂ and R₃ each independently are selected from the group consisting of hydrogen, alkyl cycloalkyl alkenyl phenyl, aryl, heteroaryl, hydroxyalkyl and mixtures thereof, and two of R₁, R₂ and R₃ may be linked to form a ring; 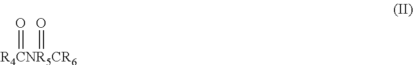
wherein R₄, R₅ and R₆ each independently are selected from the group consisting of hydrogen, alkyl, cycloalkyl, alkenyl, phenyl, aryl, heteroaryl, hydroalkyl and mixtures thereof, and two of R₄, R₅ and R₆ may be linked to form a ring; or
R₇—CN (III)
wherein R₇ is selected from the group consisting of alkyl, cycloalkyl, alkenyl, phenyl, aryl, heteroalkyl, hydroxyalkyl and mixtures thereof.

3. The composition according to claim 1 wherein the photo protective enhancing agent has a molecular weight of no higher than 300.

4. The composition according to claim 1 wherein the dielectric constant ranges from 7.5 to 12.

5. The composition according to claim 1 wherein the water-insoluble UVA organic sunscreen is Avobenzone or Benzophenone-3.

6. The composition according to claim 1 wherein the photo protective enhancing agent is a phthalimide or a dialkyldecamide.

7. The composition according to claim 6 wherein the phthalimide is butyl isopropyl-phthalimide.

8. The composition according to claim 6 wherein the dialkyldecamide is dimethyldecamide.
